(19) United States
(12) Patent Application Publication
(10) Pub. No.: US 2010/0174089 A1
(43) Pub. Date: Jul. 8, 2010

(54) HETEROARYL ARYL ETHERS

(75) Inventors: Michael Kreis, Leverkusen (DE); Edith Beckmann, Köln (DE)

Correspondence Address: LANXESS CORPORATION, 111 RIDC PARK WEST DRIVE, PITTSBURGH, PA, 15275-1112, US

(73) Assignee: SALTIGO GMBH, Langenfeld (DE)

(21) Appl. No.: 12/579,568

(22) Filed: Oct. 15, 2009

(30) Foreign Application Priority Data
Oct. 25, 2008 (DE) 10 2008 053 242.8

(51) Int. Cl. C07D 213/63; A61K 8/00

(52) U.S. Cl. 546/290

(57) ABSTRACT

The present invention relates to a process for the preparation of optionally substituted heteroaryl aryl ethers, in particular of phenoxypyridines.

### BACKGROUND OF THE INVENTION

The present invention relates to a process for the preparation of optionally substituted heteroaryl aryl ethers, in particular of phenoxypyridines.

Heteroaryl aryl ethers are of great importance as fine chemicals and intermediates for the production of medicaments, such as e.g. antidepressants, antibiotics or serotonin reuptake inhibitors and agrochemicals.

Various processes for the preparation of heteroaryl aryl ethers are known.

Their synthesis can take place, for example, via the reaction of pyridylpyridinium salts with phenols using bases (Chem. Ber. (1956), 89, 2921-2933; JP 2001002644 A). A disadvantage of this process is that pyridylpyridinium salts firstly have to be condensed from the corresponding chlorinated pyridines and then pyridine is produced as unusable by-product. Consequently, this process is ecologically and economically disadvantageous.

Cherng et al. (Tetrahedron 58 (2002), 4931-4935) describe the preparation of heteroaryl aryl ethers by substitution of halopyridines with nucleophiles in polar solvents with microwave irradiation. A disadvantage of this process is that to achieve good yields expensive starting materials, such as e.g. 4-iodopyridine, have to be used and only poor yields are obtained.

Angelo et al. (Tetrahedron Letters 47 (2006) 5045-5048) describe the reaction of chlorine heterocycles with phenol derivatives with microwave irradiation in the presence of copper powder as catalyst and cesium carbonate as base for the preparation of substituted heteroaryl aryl ethers. On account of the toxicity of the copper, and also the use of microwave heating and the expensive cesium carbonate, the process is not suitable for industrial use.

DE 69829048 T2 describes a process which leads to 4-(4-pyridinoxy)benzaldehyde by substitution of 4-chloropyridine hydrochloride with 4-hydroxybenzaldehyde using potassium carbonate as base in N,N-dimethylformamide. A disadvantage of this process is that only small yields of at most 13% of theory are obtained.

DE 60201819 T2 describes the preparation of heteroaryl aryl ethers by substitution of pyridinylene by phenolates under mild conditions. A disadvantage of this process is likewise the low yield and the limitation of the starting materials to pyridinylene substituted by electron-donating radicals.

It is common to the above processes that they either do not produce good yields or require expensive speciality chemicals and are consequently unsuitable for the industrial production of heteroaryl aryl ethers. There was consequently the need to provide a process which is suitable for the efficient preparation of heteroaryl aryl ethers.

Surprisingly, it has now been found that the reaction of an optionally substituted phenol with an optionally substituted heteroaryl halide or heteroaryl pseudohalide under suitable reaction conditions proceeds with high chemical yields to give a heteroaryl aryl ether.

### SUMMARY OF THE INVENTION

The invention therefore provides a process for the preparation of compounds of the formula (I)

ARYL-O-HETEROARYL (I)

where ARYL is C₆-C₂₀-aryl which is optionally mono- or polysubstituted by radicals which are selected, independently of one another, from the group
alkyl, alkenyl, alkoxy, alkoxycarbonyl, alkoxycarbonylamino, dialkylamino, aryl, arylalkyl, halogen, haloalkyl, haloalkylene, haloalkoxy, haloalkylthio, 5- to 6-membered heteroaryl, and 3- to 7-membered saturated or partially unsaturated heterocycle
and HETEROARYL is pyrazinyl, pyridyl, pyrimidinyl or pyridazinyl which is optionally mono- or polysubstituted by radicals which are selected, independently of one another, from the group alkyl, alkenyl, alkoxy, alkoxycarbonyl, alkoxycarbonylamino, dialkylamino, aryl, arylalkyl, halogen, haloalkyl, haloalkylene, haloalkoxy, haloalkylthio, 5- to 6-membered heteroaryl, 3- to 7-membered saturated or partially unsaturated heterocycle
which is characterized in that compounds of the formula (II)

ARYL-O⁻Cat⁺ (II)

where ARYL has the aforementioned meaning and Cat⁺ is any desired singly charged cation or a 1/nth equivalent of an n-valent cation,
are reacted with a compound of the formula (III)

HETEROARYL-Y (III)

where HETEROARYL has the aforementioned meaning and Y is a halogen or pseudohalogen.

The scope of the invention encompasses all radical definitions, parameters and illustrations above and listed hereinbelow, specified in general or within areas of preference, in any combination with one another, i.e. also between the particular areas and areas of preference.

Within the context of the invention, alkyl or alkenyl or alkoxy is a straight-chain, cyclic, branched or unbranched alkyl or alkenyl or alkoxy radical having 1 to 15 or 2 to 6 or having 1 to 6 carbon atoms.

By way of example and preferably, alkyl is methyl, ethyl, n-propyl, isopropyl, n-, iso-, s- or t-butyl, cyclopropyl, cyclobutyl, cyclopentyl, cyclohexyl, cycloheptyl, n-pentyl, 1-methylbutyl, 2-methylbutyl, 3-methylbutyl, neopentyl, 1-ethylpropyl, cyclohexyl, cyclopentyl, n-hexyl, n-heptyl, n-octyl, n-decyl and n-dodecyl.

By way of example and preferably, alkenyl is vinyl, allyl, isopropenyl and n-but-2-en-1-yl.

By way of example and preferably, alkoxy is methoxy, ethoxy, n-propoxy, isopropoxy, t-butoxy, n-pentoxy and n-hexoxy.

Within the context of the invention, alkoxycarbonyl is preferably a straight-chain or branched alkoxy radical having 1 to 6 carbon atoms which is linked via a carbonyl group. The following may be mentioned by way of example and preferably: methoxycarbonyl, ethoxycarbonyl, n-propoxycarbonyl, isopropoxycarbonyl and t-butoxycarbonyl.

Within the context of the invention, alkoxycarbonylamino is an amino group with a straight-chain or branched alkoxy carbonyl substituent which preferably has 1 to 6 carbon atoms in the alkoxy radical and is linked via the carbonyl group. The following may be mentioned by way of example and preferably: methoxycarbonylamino, ethoxycarbonylamino, n-propoxycarbonylamino and t-butoxycarbonylamino.

Within the context of the invention, aryl is a mono-, bi- or tricyclic carbocyclic aromatic radical having preferably 6 to 20 aromatic carbon atoms (C₆-C₂₀-aryl). Furthermore, the carbocyclic aromatic radicals can be substituted by up to five identical or different substituents per cycle, selected from the group alkyl, alkenyl, alkoxy, alkoxycarbonyl, alkoxycarbonylamino, aryl, arylalkyl, dialkylamino, halogen, haloalkyl, haloalkylene, haloalkoxy, haloalkylthio, 5- to 6-membered heteroaryl and 3- to 7-membered saturated or partially unsaturated heterocycle. By way of example and preferably, C₆-C₂₀-aryl is biphenyl, phenyl, naphthyl, phenanthrenyl, anthracenyl or fluorenyl.

Arylalkyl means in each case independently of one another a straight-chain, cyclic, branched or unbranched alkyl radical according to the above definition, which can be monosubstituted, polysubstituted or completely substituted by aryl radicals according to the above definition. One example of arylalkyl is benzyl.

By way of example and preferably, halogens are fluorine, chlorine or bromine, particularly preferably chlorine.

Within the context of the invention, dialkylamino is an amino group having one or two identical or different, cyclic, straight-chain or branched alkyl substituents which preferably in each case have 1 to 6 carbon atoms.

By way of example and preferably, dialkylamino is N,N-dimethylamino, N,N-diethylamino, N-ethyl-N-methylamino, N-methyl-N-n-propylamino, N-Isopropyl-N-n-propylamino, N-t-butyl-N-methylamino, N-ethyl-N-n-pentylamino and N-n-hexyl-N-methylamino.

Within the context of the invention, haloalkyl or haloalkylene or haloalkoxy is a straight-chain, cyclic, branched or unbranched alkyl or alkylene or alkoxy radical according to the above definition which is monosubstituted, polysubstituted or completely substituted by halogen atoms.

By way of example and preferably, haloalkyl is dichloromethyl, difluoromethyl, fluoromethyl, trifluoromethyl, trichloromethyl, 2,2,2-trifluoroethyl, pentafluoroethyl, heptafluoroisopropyl and nonafluorobutyl.

By way of example and preferably, haloalkylene is chloroethylene, dichloroethylene or trifluoroethylene.

By way of example and preferably, haloalkoxy is difluoromethoxy, fluoroethoxy, fluoromethoxy, trifluoromethoxy, trichloromethoxy and 2,2,2-trifluoroethoxy.

Within the context of the invention, haloalkylthio is a straight-chain, cyclic, branched or unbranched radical having 1 to 15 carbon atoms which is monosubstituted, polysubstituted or completely substituted by halogen atoms. By way of example and preferably, haloalkylthio is chloroethylthio, chlorobutylthio, chlorohexylthio, chloropentylthio, chlorododecylthio, dichloroethylthio, fluoroethylthio, trifluoromethylthio and 2,2,2-trifluoroethylthio.

Within the context of the invention, 5- to 6-membered heteroaryl is preferably an aromatic heterocycle with up to 3 identical or different heteroatoms from the series S, N and/or O which is linked via a ring carbon atom of the heteroaromatic, optionally also via a ring nitrogen atom of the heteroaromatic. By way of example, the following may be mentioned: furanyl, pyrrolyl, thienyl, thiazolyl, oxazolyl, imidazolyl, triazolyl, pyridyl, pyrimidinyl, pyridazinyl. Preference is given to pyridyl, pyrimidinyl, pyridazinyl, furyl and thiazolyl.

Within the context of the invention, 3- to 7-membered saturated or partially unsaturated heterocycle is preferably a heterocycle with up to 3 identical or different heteroatoms from the series S, N and/or O which is linked via a ring carbon atom or a ring nitrogen atom and which can contain one or two double bonds. Preference is given to a 5- to 7-membered saturated heterocycle with up to 2 identical or different heteroatoms from the series S, N and/or O. By way of example, the following may be mentioned: tetrahydrofur-2-yl, tetrahydrofur-3-yl, pyrrolidin-1-yl, pyrrolidin-2-yl, pyrrolidin-3-yl, pyrrolin-1-yl, piperidin-1-yl, piperidin-4-yl, 1,2-dihydropyridin-1-yl, 1,4-dihydropyridin-1-yl, piperazin-1-yl, morpholin-4-yl, thiomorpholin-4-yl, azepin-1-yl, 1,4-diazepin-1-yl. Preference is given to piperidinyl, piperazinyl, morpholinyl and pyrrolidinyl.

Within the context of the invention, pseudohalogen refers to radicals whose chemical properties are very similar to those of the halogens. These are e.g. sulfonates and halosulfonates, such as e.g. tosylate, triflate, mesylate and nonafluorobutylsulfonate, but also thiocyanate and azide.

Preferably, ARYL is a C₆-C₂₀-aryl radical which is optionally monosubstituted or polysubstituted by radicals which are selected, independently of one another, from the group alkoxy, dialkylamino, haloalkyl, haloalkylthio or haloalkyloxy.

In a particularly preferred embodiment, ARYL is a C₆-C₂₀-aryl radical which is optionally monosubstituted or polysubstituted by radicals which are selected, independently of one another, from the group trifluoromethoxy, methoxy and methyl. In a very particularly preferred embodiment, ARYL is a C₆-C₂₀-aryl radical which is optionally monosubstituted or polysubstituted by radicals from the group trifluoromethoxy, methoxy and methyl in the 2, 3 and/or 4 position.

In one preferred embodiment, HETEROARYL is pyridyl which is optionally monosubstituted or polysubstituted by radicals which are selected, independently of one another, from the group alkoxy, alkyl, aryl, haloalkyl, haloalkylthio or haloalkyloxy. In a very particularly preferred embodiment, HETEROARYL is pyridyl which is optionally monosubstituted or polysubstituted by radicals from the group trifluoromethoxy, methoxy and methyl in the 2, 3 and/or 4 position.

Preferred compounds of the formula (II) are phenol, 4-methoxyphenol, 2-trifluoromethoxyphenol, 4-trifluoromethoxyphenol and p-kresol. A preferred compound of the formula (III) is 4-chloropyridine. In a further particularly preferred embodiment, the compounds of the formula (I) are 4-[4-trifluoromethoxyphenoxy]pyridine, 4-[4-methylphenoxy]pyridine, 4-phenoxypyridine, 4-[4-methoxyphenoxy]pyridine and 4-[2-trifluoromethoxyphenoxy]pyridine.

The compounds of the formula (III) can be prepared, for example, by reacting compounds of the formula (IIIa)

HETEROARYL-Y*HX (IIIa)

with a base. HX is a protic acid. By way of example and preferably, the compound (Ma) is a salt of the compound (III) in the form of the hydrochloride, hydrobromide, hydroiodide, hydrogen sulphate or hydrofluoride.

Within the context of the process according to the invention, the compounds of the formula (II) can be prepared, for example, by reacting compounds of the formula (IIa)

ARYL-OH (IIa)

with a suitable base which is able to deprotonate the phenols used. Preferably, the corresponding acids of these bases have a pKa value of >10, measured under standard conditions. The pKa value is particularly preferably >15.

Examples of suitable bases which may be listed here are alkaline earth metal or alkali metal hydrides, hydroxides, amides, alcoholates or carbonates, such as, for example, sodium hydride, sodium amide, lithium diethylamide, sodium methylate, potassium tert-butylate, sodium hydroxide, potassium hydroxide, sodium carbonate, potassium carbonate, and also tertiary amines, such as trimethylamine, triethylamine, tributylamine, diisopropylethylamine, N,N-dimethylaniline, piperidine, N-methylpiperidine, N,N-dimethylaminopyridine and 1,5-diazabicyclo[4.3.0]non-5-ene (DBN), 1,8-diazabicyclo[5.4.0]undec-7-ene (DBU), 1,1,3,3-tetramethylguanidine (TMG), 7-methyl-1,5,7-triazabicyclo[4.4.0]dec-5-ene (MTBD) and 2,8,9-triisopropyl-2,5,8,9-tetraaza-1-phosphabicyclo[3.3.3]undecane (TTPU). Further examples of suitable bases are aryl anions and cyclopentadienyl anions. Particular preference is given to the use of alkaline earth metal or alkali metal hydroxides, and also sterically hindered alcoholates such as e.g. potassium tert-butylate.

During the industrial production of compounds of the formula (I), polymerization products of compounds of the formula (III) are undesired by-products, which should largely be avoided. These polymerization products are formed, for example, by autocondensation of the compound of the formula (III) in the storage vessel or in the initial charge at high temperatures and lead to reduced purities and yields and can, moreover, block pipelines. Consequently, a special procedure is advantageous.

The process is therefore preferably carried out by, for example, firstly initially introducing the compound of the formula (II), optionally in one or more solvents and optionally in the presence of one or more suitable bases, and then adding the compound of the formula (III). Alternatively, the compound of the formula (IIa), optionally in one or more solvents, can be initially introduced and, in the presence of one or more suitable bases, be brought into contact with the compound of the formula (III). Alternatively, the compound of the formula (IIa) or of the formula (II), optionally in one or more solvents, can be initially introduced, be admixed with an excess of base and be brought into contact with the compound of the formula (IIIa). Alternatively, the reaction can also be carried out with compounds of the formula (IIIa) and base and more precisely in such a way that the compounds of the formula (III) are, for example, firstly prepared from compounds of the formula (IIIa) by adding suitable bases.

In a further embodiment, the deprotonation of the formula (IIIa) can also take place in situ.

Suitable solvents for carrying out the process according to the invention are, in particular, organic solvents. Suitable organic solvents are, for example, aliphatic, alicyclic or aromatic, optionally halogenated hydrocarbons, such as, for example, benzene, toluene, xylene, various petroleum ethers, hexane, cyclohexane, tetrachloromethane; ethers, such as diethyl ether, methyl tert-butyl ether, diisopropyl ether, dioxane, tetrahydrofuran or ethylene glycol dimethyl or diethyl ether; ketones, such as acetone, 2-butanone or methyl isobutyl ketone; amides, such as N,N-dimethylformamide, N,N-dimethylacetamide, N-methylformanilide, N-methylpyrrolidone, N-methylcaprolactam or hexamethylphosphoric acid triamide, sulphoxides, such as dimethyl sulphoxide, sulfones such as tetramethylenesulfone, or mixtures of such organic solvents. Preference is given to aromatic hydrocarbons, aliphatic and alicyclic amides, sulphoxides and sulpholanes, particular preference being given to N,N-dimethylacetamide, N,N-dimethylformamide, p-xylene and xylene isomer mixture or mixtures.

The reaction temperature can be, for example, between 20° C. and 300° C. The reaction temperature is preferably between 125° C. and 200° C., particularly preferably between 135° C. and 180° C.

For example, the temperature of the compound of the formula (III) in the storage vessel can be between −30° C. and 20° C. Consequently, the autocondensation in the storage vessel is essentially prevented.

The process according to the invention is preferably carried out essentially free from transition metals of groups 4 to 12 of the Periodic Table of the Elements, such as in particular copper. The term “essentially free” means a content of transition metal based on the sum of the mass of the compounds of the formula (II) and of the formula (III) to be coupled of from 0 to 1000 ppm, preferably 0-10 ppm.

In principle, it is possible to work under variable pressure. Preference is given to working at ambient pressure.

Preferably, the addition of the compound of the formula (III) or of the formula (IIIa) is carried out such that the ratios of the quantitative amounts of the compound of the formula (II) and of the compound of the formula (III) or of the formula (IIIa) during the addition are between 5:1 and 1000:1. The addition can take place, for example, in portions, semicontinuously or continuously. The addition takes place particularly preferably in portions. The ratio of the quantitative amounts of the compound of the formula (II) to the compound of the formula (III) or of the formula (IIIa) during the addition is particularly preferably between 5:1 and 20:1.

For example, the quantitative amount ratio of the compounds of the formula (II) used and of the compounds of the formula (III) or of the formula (IIIa), based on the total reaction, can be between 1:2 and 10:1, preferably between 1:1 and 5:1, particularly preferably between 1:1 and 2:1.

In one particularly preferred embodiment, the compound of the formula (IIa) is brought into contact with the suitable base. The initial charge is then heated to the reaction temperature. The compound of the formula (IIIa) is then added in portions such that the quantitative amount ratio of the compound of the formula (II) to the compound of the formula (IIIa) during the addition is between 5:1 and 20:1. In one preferred embodiment, the reaction temperature is between 135° C. and 180° C.

In the manner according to the invention, it is possible to prepare the compounds of the formula (I) in high yields in industrial processes. The work-up can take place in a manner known per se, e.g. by extraction with known solvents such as, for example, water and methyl tert-butyl ether.

The compounds of the formula (I) prepared according to the invention are particularly suitable as intermediates e.g. for the production of fine chemicals, medicaments, such as e.g. antidepressants, antibiotics or serotonin reuptake inhibiters, and agrochemicals.

### EXAMPLES

### 1. Synthesis of 4-[4-trifluoromethoxyphenoxy]pyridine

36.4 g of potassium tert-butylate (0.32 mol) were dissolved in 200 ml of N,N-dimethylacetamide at room temperature under an inert atmosphere. 35.3 g of 4-trifluoromethoxyphenol (0.19 mol) were then added to the stirred solution over the course of 40 minutes. The solution was then heated to 100° C. and 20 g of 4-chloropyridine hydrochloride (0.13 mol) were added to the reaction solution in 4 portions over the course of 2 hours. The reaction solution was heated to 140° C. and stirred for 24 hours at 140° C. 5.0 g of potassium tert-butylate (0.045 mol) were added and the reaction was stirred for a further 16 hours at reflux. The reaction solution was cooled to room temperature and admixed with 100 ml of water and 100 ml of methyl tert-butyl ether and adjusted to pH 1 to 2 using 10% strength hydrochloric acid. The phases were separated. The aqueous phase was adjusted to pH 11 with 50 ml of 15% strength sodium hydroxide solution and extracted 3 times with in each case 100 ml of methyl tert-butyl ether. The combined organic phases were washed with 500 ml of water. The solvent was removed under reduced pressure. This gave 25.3 g of 4-[4-trifluoromethoxyphenoxy]pyridine with a purity of 85.5% by weight (0.085 mol) and a yield of 65.5 mol % of theory.

### 2. Synthesis of 4-[4-methylphenoxy]pyridine

40.4 g of potassium tert-butylate (0.36 mol) were dissolved in 220 g of N,N-dimethylacetamide at room temperature under inert atmosphere. 19.5 g (0.18 mol) of p-cresol were then added to the stirred solution over the course of 40 minutes. The solution was then heated to 140° C., and 18 g of 4-chloropyridine hydrochloride (0.12 mol) were added to the reaction solution in 10 portions at intervals of 30 minutes. The reaction solution was stirred for 60 hours at 140° C. The reaction solution was cooled to room temperature and admixed with 100 ml of water and 150 ml of methyl tert-butyl ether and adjusted to pH 1 using 37% strength hydrochloric acid. The phases were separated. The aqueous phase was adjusted to pH >11 using 50% strength sodium hydroxide solution and extracted twice with in each case 150 ml of methyl tert-butyl ether. The organic phases were combined. The solvent was removed under reduced pressure. This gave 20.5 g of 4-[4-methylphenoxy]pyridine (0.11 mol, 91 mol % of theory).

### 3. Synthesis of 4-phenoxypyridine

75 g of potassium tert-butylate (0.63 mol) were dissolved in 375 g of N,N-dimethylacetamide at room temperature under inert atmosphere. 30 g (0.32 mol) of phenol were then added to the stirred solution over the course of 40 minutes. The solution was then heated to 140° C., and 32 g of 4-chloropyridine hydrochloride (0.21 mol) were added to the reaction solution in portions over the course of 5 h. The reaction solution was stirred for 19 h at 140° C. The reaction solution was cooled to room temperature and admixed with 100 ml of water and 150 ml of methyl tert-butyl ether and adjusted to pH 1 using 37% strength hydrochloric acid. The phases were separated. The aqueous phase was adjusted to pH>11 using 50% strength sodium hydroxide solution and extracted twice using in each case 150 ml of methyl tert-butyl ether. The organic phases were combined. The solvent was removed under reduced pressure. This gave 34 g of phenoxypyridine (0.19 mol, 94 mol % of theory).

### 4. Synthesis of 4-[4-methoxyphenoxy]pyridine

58.0 g of potassium tert-butylate (0.52 mol) were dissolved in 232.3 g of N,N-dimethylacetamide at room temperature under inert atmosphere. 48.2 g (0.39 mol) of 4-methoxylphenol were then added to the stirred solution. The solution was then heated to 136-140° C., and 40 g of chloropyridine hydrochloride (0.26 mol) were metered in in 8 portions over the course of 18 h. The reaction solution was then stirred for 7 h at 140° C. The reaction solution was cooled to room temperature and admixed with 325 g of water and 300 g of methyl tert-butyl ether and adjusted to pH 1 using 37% strength hydrochloric acid. The phases were separated. The aqueous phase was adjusted to pH>11 using 50% strength sodium hydroxide solution and extracted twice with in each case 200 g of methyl tert-butyl ether. The organic phases were combined. The solvent was removed under reduced pressure. This gave 45.5 g of 4-[4-methoxyphenoxy]pyridine (0.23 mol, 87 mol % of theory).

### 5. Synthesis of 4-[2-trifluoromethoxyphenoxy]pyridine

16.7 g of potassium tert-butylate (0.15 mol) were dissolved in 67 g of N,N-dimethylacetamide at room temperature under inert atmosphere. 19.9 g (0.11 mol) of 2-trifluoromethoxyphenol were then added to the stirred solution. The solution was then heated to 140° C., and 11.2 g of chloropyridine hydrochloride (0.075 mol) were added to the reaction mixture in 10 portions at intervals of 30 minutes and stirred for 60 h at 140° C. The reaction solution was cooled to room temperature and admixed with 150 ml of water and 150 ml of methyl tert-butyl ether and adjusted to pH 1 using 37% strength hydrochloric acid. The phases were separated. The aqueous phase was adjusted to pH>11 using 50% strength sodium hydroxide solution and extracted twice with in each case 150 ml of methyl tert-butyl ether. The organic phases were combined. The solvent was removed under reduced pressure. This gave 14.2 g of 4-[2-trifluoromethoxyphenoxy]pyridine (0.057 mol, 75 mol% of theory).

What is claimed is:

1. A process for preparing a compound of the formula (I)
ARYL-O-HETEROARYL (I)
wherein ARYL is C₆-C₂₀-aryl
and
HETEROARYL is pyrazinyl, pyridyl, pyrimidinyl or pyridazinyl comprising reacting compounds of the formula (II)
ARYL-O⁻Cat⁺ (II)
wherein ARYL has the aforementioned meaning and Cat⁺ is any desired singly charged cation or a 1/nth equivalent of an n-valent cation,
with a compound of the formula (III)
HETEROARYL-Y (III)
wherein HETEROARYL has the aforementioned meaning and Y is a halogen or pseudohalogen.

2. A process according to claim 1, wherein ARYL is C₆-C₂₀-aryl which is monosubstituted or polysubstituted by radicals which are selected, independently of one another, from the group alkyl, aryl, alkoxy, haloalkyl, haloalkoxy and haloalkylthio.

3. A process according to claim 1, wherein ARYL is C₆-C₂₀-aryl being monosubstituted or polysubstituted by radicals selected independently of one another from the group trifluoromethoxy, methoxy and methyl.

4. A process according to claim 1, wherein HETEROARYL is pyridyl being monosubstituted or polysubstituted by radicals selected independently of one another, from the group alkyl, aryl, alkoxy, haloalkyl, haloalkoxy and haloalkylthio.

5. A process according to claim 1, wherein HETEROARYL is 4-pyridyl being mono- or polysubstituted by radicals selected independently of one another, from the group trifluoromethoxy, methoxy and methyl.

6. A process according to claim 1, wherein HETEROARYL-Y is 4-chloropyridine.

7. A process according to claim 1, wherein the compound of the formula (I) is 4-[4-trifluoromethoxyphenoxy] pyridine, 4-[4-methylphenoxy]pyridine, 4-phenoxypyridine, 4-[4-methoxyphenoxy]pyridine or 4-[2-trifluoromethoxyphenoxy]-pyridine.

8. A process according to claim 1, wherein the compound of the formula (III) or of the formula (IIIa) is added to the compound of the formula (II) with the ratio of the quantitative amount of compound of the formula (II) to compounds of the formula (III) or of the formula (IIIa) during the addition is between 5:1 and 1000:1.

9. A process according to claim 1, wherein the ratio of the quantitative amount of compounds of the formula (II) to compounds of the formula (III) or of the formula (IIIa) during the addition is between 5:1 and 20:1.

10. A process to claim 1, carried out at a temperature between 20° C. and 300° C.

11. A process to claim 1, carried out at a temperature between 135° C. and 180° C.

12. A process according to claim 1, carried out essentially free from transition metals of groups 4 to 12 of the Periodic Table of the Elements.

13. A process according to claim 1, wherein the reaction takes place in the presence of N,N-dimethylacetamide, N,N-dimethylformamide, p-xylene or xylene isomer mixture.

14. A process according to claim 1, wherein the preparation of the compound (II) from the compound (IIa) is carried out using suitable bases whose corresponding acids have a pKa value of >10.
